(19) United States
(12) Patent Application Publication
(10) Pub. No.: US 2005/0129742 A1
(43) Pub. Date: Jun. 16, 2005

(54) Antimicrobial article with diffusion control layer

(75) Inventors: Joseph F. Bringley, Rochester, NY (US); Yannick J. Lerat, Chalon Sur Saone (FR); Nancy B. Liebert, Rochester, NY (US); David L. Patton, Webster, NY (US); Richard W. Wien, Pittsford, NY (US)

Correspondence Address: Paul A. Leipold;Patent Legal Staff, Eastman Kodak Company, 343 State Street, Rochester, NY, 14650-2201, US

(73) Assignee: Eastman Kodak Company

(21) Appl. No.: 10/736,974

(22) Filed: Dec. 16, 2003

(51) Int. Cl. A61K 9/70; B22F 7/02; B23K 35/36

(52) U.S. Cl. 424/443; 424/618; 428/549

(57) ABSTRACT

This invention relates to an article comprising on the surface thereof an antimicrobial layer comprising a binder and an antimicrobial compound, wherein said antimicorbial compound or an antimicrobial moiety thereof, is released into the surrounding environment; and a diffusion layer; wherein the antimicrobial layer is between the surface of the article and the diffusion layer and wherein the diffusion layer changes the rate at which the antimicrobial compound is released from the antimicrobial layer into the surrounding environment.

### FIELD OF THE INVENTION

The present invention relates to an antimicrobial article having a controlled release of an antimicrobial compound, it further relates to an article comprising a diffusion control layer that controls the rate of release of the antimicrobial compound.

### BACKGROUND OF THE INVENTION

In recent years people have become very concerned about exposure to the hazards of microbe contamination. For example, exposure to certain strains of Escherichia coli through the ingestion of under-cooked beef can have fatal consequences. Exposure to Salmonella enteritidis through contact with unwashed poultry can cause severe nausea. Mold and yeast (Candida albicans) may cause skin infections. In some instances, biocontamination alters the taste of the food or drink or makes the food unappetizing. With the increased concern by consumers, manufacturers have started to produce products having antimicrobial properties. A wide variety of antimicrobial materials have been developed which are able to slow or even stop microbial growth; such materials when applied to consumer items may decrease the risk of infection by micro-organisms.

Noble metal ions such as silver and gold ions are known for their antimicrobial properties and have been used in medical care for many years to prevent and treat infection. In recent years, this technology has been applied to consumer products to prevent the transmission of infectious disease and to kill harmful bacteria such as Staphylococcus aureus and Salmonella. In common practice, noble metals, metal ions, metal salts or compounds containing metal ions having antimicrobial properties may be applied to surfaces to impart an antimicrobial property to the surface. If, or when, the surface is inoculated with harmful microbes, the antimicrobial metal ions or metal complexes, if present in effective concentrations, will slow or even prevent altogether the growth of those microbes. Antimicrobial activity is not limited to noble metals but is also observed in organic materials such as chlorophenol compounds (Triclosan™), isothiazolone (Kathon™), antibiotics, and some polymeric materials.

It is important that the antimicrobially active element, molecule or compound, be present on the surface of the article at a concentration sufficient to inhibit microbial growth. This concentration, for a particular antimicrobial agent and bacterium, is often referred to as the minimum inhibitory concentration (MIC). It is also important that the antimicrobial agent be present on the surface of said article at a concentration significantly below that which may be harmful to the user of said article. This prevents harmful side effects of the article and decreases the risk to the user, while still providing the benefit of reducing microbial contamination. More recently, metal ion exchange materials have been developed which are able to effect the so-called “controlled release” of an antimicrobial ion, by virtue of exchange of the antimicrobial ion with ions commonly present in biological environments. This approach is very general since innocuous ions such as sodium and potassium are present in virtually all biological environments. The approach has the advantage in that the antimicrobial ions are bound tightly by the ion exchange medium, but are released when exposed to conditions under which biological growth may occur.

U.S. Patent application 0091767 A1 to Podhajny, describes a method of applying an antimicrobial treatment to a packaging material and polymer dispersions containing antimicrobial zeolites. The polymeric dispersions contain zeolites, which release antimicrobial metal ions, such as silver, and may be formulated in water-based or solvent-based systems. Suitable polymers for practice of the invention listed are polyamides, acrylics, polyvinyl chloride, polymethyl methacrylates, polyurethane, ethyl cellulose and nitro celluloses.

U.S. Pat. No. 5,556,699 to Niira et al. describes transparent polymeric films containing antimicrobial zeolites which are ion exchanged with silver and other ions. The films are said to display antimicrobial properties. Polymeric materials suitable for the invention include ethylene ethyl acrylate (EEA), ethylene vinyl acetate (EVA), polyethylene, polyvinyl chlorides, polyvinyl fluoride resins and others.

U.S. Pat. No. 6,626,873 B1 to Modak et al. describes polymeric medical articles comprising the anti-infective agents chlorhexidine and triclosan. It further describes a polymeric medical article impregnated with a treatment solution comprising (i) between about 1 and 10 percent of a hydrophilic polymer; (ii) between 1 and 5 percent of chlorhexidine; and between 0.5 and 5.0 percent of triclosan.

### Problem to be Solved by the Invention

There is a problem in that the polymeric binder or polymeric medium may severely limit the release of the antimicrobial material. Therefore, the exchange of antimicrobial ions from the antimicrobial films may not be facile enough to achieve a concentration of antimicrobial metal ions sufficient to limit the growth rate of a particular microbe, or may not be above the minimum inhibitory concentration (MIC). Alternatively, there is a problem in that the rate of release of antimicrobial ions from antimicrobial films may be too facile, such that the antimicrobial film may quickly be depleted of antimicrobial active materials and become inert or non-functional. Depletion results from rapid diffusion of the active materials into the biological environment with which they are in contact. It is desirable that the rate of release of the antimicrobial ions or molecules be controlled such that the concentration of antimicrobials remains above the MIC. The concentration should remain there over the duration of use of the antimicrobial article. The desired rate of exchange of the antimicrobial may depend upon a number of factors including the identity of the antimicrobial metal ion, the specific microbe to be targeted, and the intended use and duration of use of the antimicrobial article.

There remains a need to control the release of an antimicrobial active compound from an article, such that a minimum inhibitory concentration of the antimicrobial compound may be achieved at the surfaces of the article for the duration of the use of said article, under the common operating environment of said article. There remains a further need to control the release of an antimicrobial active material from an article, such that the antimicrobially active material is not released too quickly, especially at levels significantly beyond the minimum inhibitory concentration, so that the activity of the article is long lasting. There is a further need for antimicrobial articles which are simple to formulate, and that have excellent physical properties such as resistance to scratching, staining, abrasion, etc.

### SUMMARY OF THE INVENTION

This invention provides an article comprising on the surface thereof an antimicrobial layer comprising a binder and an antimicrobial compound which is released into the surrounding environment; and a diffusion layer; wherein the antimicrobial layer is between the surface of the article and the diffusion layer and wherein the diffusion layer changes the rate at which the antimicrobial compound is released from the antimicrobial layer into the surrounding environment.

It further provides a multilayer medium having antimicrobial properties comprising a support, an antimicrobial layer comprising a binder and an antimicrobial compound which is released into the surrounding environment; and a diffusion layer; wherein the antimicrobial layer is between the support and the diffusion layer and wherein the diffusion layer changes the rate at which the antimicrobial compound is released from the antimicrobial layer into the surrounding environment.

This invention provides a useful antimicrobial article suitable for many uses. The article of the invention quickly provides a minimum inhibitory concentration of the antimicrobial metal at its surface, under the common operating environment of said article. It provides this effect for a sustained period of time even at relatively low laydowns of antimicrobial compounds. It further provides a multilayer medium which may be applied to an article to provide antimicrobial properties to the article.

### DETAILED DESCRIPTION OF THE INVENTION

Articles having antimicrobial properties may be prepared by application of an antimicrobial compound (hereafter referred to as AMC) to the surface of the article, or by embedding an AMC within the article. In most instances, microbes may reside only at the surface of an article, and thus the AMC is applied only to the surface. The AMC may be applied by many methods such as coating, spraying, casting, blowing, extruding, etc. Typically, the AMC is dissolved or dispersed in a vehicle (such as a solvent) and a binder (such as a polymer) which provides a means of adhering the AMC to the article surface. Alternatively, the AMC may be mixed or compounded directly within the polymer, and the mixture subsequently melted and extruded to form a film. The film may then be attached to an article by means such as gluing or lamination.

Upon use and exposure of an antimicrobial article to conditions under which microbial growth may occur, the AMC may then leach from the surface of the article to kill or inhibit the growth of microbes present thereon. In some cases only a portion (antimicrobial moiety) of the antimicrobial compound may leach into the surrounding environment, e.g., in the case of an antimicrobial metal ion exchange material only the antimicrobial metal ion (antimicrobial moiety) is released. The following discussion regarding the diffusion of AMCs is also applicable to an antimicrobial moiety. In order for the article to have antimicrobial properties, the AMC must leach out at a rate fast enough to establish and maintain a minimum inhibitory concentration (MIC). Below the MIC, microbial growth may continue uninhibited. Likewise, it is important that the AMC not leach out so fast as to quickly deplete the article of AMC and thus limit the longevity of the effectiveness of the article. The rate at which the AMC may leach (or diffuse) is dependent upon its degree of solubilization in aqueous media (water). This is an essential point, since microbial growth requires high water activity commonly found in wet or humid environments. Because most antimicrobial materials are substantially soluble in water, the rate of diffusion of the AMC will be limited by the rate at which water can diffuse to the AMC and hence dissolve it. This is especially true for solid-phase AMC's, since diffusion may not occur until the AMC is dissolved or solubilized. If the AMC is embedded in a polymer which very quickly adsorbs water, the article may be quickly depleted of antimicrobial activity, since the AMC contained at its surface may quickly leach into the surrounding environment via the solubilization mechanism discussed above. Alternatively, if the AMC is embedded in a polymer that does not adsorb water, or only adsorbs water extremely slowly, then the AMC may diffuse very slowly or not at all, and a MIC may never be achieved in the surrounding environment. A measure of the permeability of various polymeric addenda to water is given by the permeability coefficient, P which is given by
P=(quantity of permeate)(film thickness)/[area×time×(pressure drop across the film)]
Permeability coefficients and diffusion data of water for various polymers are discussed by J. Comyn, in Polymer Permeability, Elsevier, N.Y., 1985 and in “Permeability and Other Film Properties Of Plastics and Elastomers”, Plastics Design Library, NY, 1995. The higher the permeability coefficient, the greater the water permeability of the polymeric media. The permeability coefficient of a particular polymer may vary depending upon the density, crystallinity, molecular weight, degree of cross-linking, and the presence of addenda such as coating-aids, plasticizers, etc.

The article of the invention comprises on the surface thereof an antimicrobial layer comprising a binder and an antimicrobial compound, wherein said antimicorbial compound or an antimicrobial moiety of the antimicrobial compound is released into the surrounding environment. It further comprises a diffusion layer wherein the antimicrobial layer is between the surface of the article and the diffusion layer. The diffusion layer changes the rate at which the antimicrobial compound or moiety is released from the antimicrobial layer into the surrounding environment. The “surrounding environment” may include a thin film of water contacting the surface, or any environment which is capable of supporting biological growth such as water, salt water, saliva, body fluids, food extrudates, food, etc.

The diffusion layer of the inventive article controls the rate at which the antimicrobial compound is released from the antimicrobial layer into the surrounding environment. The water permeability of the polymer of the diffusion layer is different from that of the binder comprising the antimicrobial layer. It is shown herein that the rate of leaching of an AMC into a surrounding biological environment is dependent upon the rate at which water is adsorbed by the polymeric media in which the AMC is contained. Therefore, the diffusion layer of the invention herein, by virtue of its differing water permeability, controls the rate at which the AMC is released. In a preferred embodiment the diffusion layer has a water permeability that is greater than the water permeability of the antimicrobial layer. This is preferred because it will have the effect of speeding the rate at which the AMC is released from the article, and hence a MIC may be achieved in this article quickly upon exposure to a biological environment. For example, if a highly hydrophilic polymer is employed as the diffusion layer, and this polymer is able to absorb water, for example, from moist air (such as gelatin or polyvinylalcohol) then the polymer will precondition the underlying antimicrobial layer as it will be contacted with a much greater equilibrium moisture content than if the diffusion layer where not present. In this manner some AMC is expected to leak into the diffusion layer, which, when contacted with a biological environment, will allow the AMC to leach quickly. The invention could then be suitably applied to applications that require quick release of AMC, and do not require longevity. Examples of such items are wash-cloths, paper-towels, wipes, disposable items such as paper plates, wrapping materials such as paper, waxed paper, cellophane and plastic films. In another preferred embodiment the diffusion layer has a water permeability that is less than the water permeability of the antimicrobial layer. This is preferred because it will have the effect of slowing the rate at which the AMC is released from the article. Such articles generally require that the laydown of the AMC of the antimicrobial layer be greater than the aforementioned case, and hence a MIC may be achieved at the surface of this article more slowly, but is sustained over a much longer period of time, since the rate of release will be slower. The invention could then be suitably applied to applications that require slow but sustained release of AMC. Examples of such items are counter-tops, walls, floors, rugs, textiles and clothing, medical components, items having laminated plastic thereon, household appliances and refrigerator surfaces, etc.

In another preferred embodiment, the diffusion layer has a water permeability greater than 500 [(cm³ cm)/(cm²sec/Pa)]×10¹³. This is preferred because diffusion layers having water permeabilities below this value would severely limit the diffusion of AMC to the surface and would require very high-laydowns of AMC, and would thus be expensive to produce. In still other preferred embodiments the polymer of the diffusion is selected from polyurethanes, polyesters, polyamides, polymethacrylates, polyethylene terephthalate, polyethylene-polyvinyl alcohol copolymer, polystyrene, ethyl cellulose, cellulose acetate and cellulose nitrate, polyethylene and polypropylene, nylon and polyacrylonitrile. More preferred are the polyurethanes, polyesters, polyamides, cellulose acetate, polymethacrylates, polystyrene, polypropylene, polyethylene-polyvinyl alcohol copolymer and polyethylene.

The diffusion layer may vary in thickness, however, very thick diffusion layers may severely limit the rate at which the AMC or antimicrobial moiety will be released. The required thickness may depend upon a number of factors including the required physical properties of the material, such as hardness, toughness, scratch resistance, etc. in addition to the antimicrobial requirements of the article. It is preferred that the diffusion layer has a thickness in the range of 0.1 microns to 10 microns. It is further preferred that the thickness of said diffusion layer is about 1.0 micron to 5.0 microns.

The antimicrobial layer contains at least one AMC and a binder. The binder may be a polymeric species, a latex, or an inorganic material such as a sol-gel. The primary purpose of the binder is to provide a method of attaching the AMC to the surface of the article. Another purpose of the binder is to provide a convenient and simple vehicle to handle and later apply the AMC to the surface. It is preferred that the binder be aqueous compatible, such that the antimicrobial layer be conveniently applied from water based dispersions, solutions or emulsions. It is further preferred that the antimicrobial layer has a water permeability of greater than 5000 [(cm³ cm)/(cm²sec/Pa)]×10¹³. It is still further preferred that the binder of the antimicrobial layer comprises polyvinyl alcohol, cellophane, water-based polyurethanes, nylon, high nitrile resins, polyethylene-polyvinyl alcohol copolymer, polystyrene, ethyl cellulose cellulose acetate, cellulose nitrate, aqueous latexes, polyacrylic acid, or polystyrene sulfonate.

The antimicrobial active compound of the antimicrobial composition may be selected from a wide range of known antibiotics and antimicrobials. Suitable materials are discussed in “Active Packaging of Food Applications” A. L. Brody, E. R. Strupinsky and L. R. Kline, Technomic Publishing Company, Inc. Pennsylvania (2001). Examples of antimicrobial agents suitable for practice of the invention include benzoic acid, sorbic acid, nisin, thymol, allicin, peroxides, imazalil, triclosan™, benomyl, antimicrobial metal-ion exchange materials, metal colloids, metal salts, anhydrides, and organic quaternary ammonium salts. Either the compound itself or an antimicrobial moiety released from the antimicrobial compound is preferably aqueously soluble.

In a preferred embodiment, the antimicrobial compound is selected from metal ion-exchange materials that have been exchanged or loaded with antimicrobial ions. Metal ion-exchange materials suitable for practice of the invention are selected from zirconium phosphates, metal hydrogen phosphates, sodium zirconium hydrogen phosphates, zeolites, clays such as montmorillonite, ion-exchange resins and polymers, porous alumino-silicates, layered ion-exchange materials and magnesium silicates. Preferred metal ion exchange materials are zirconium phosphate, metal hydrogen phosphate, sodium zirconium hydrogen phosphate, or zeolite. Preferred antimicrobial ions are silver, copper, nickel, zinc, tin and gold. In a particularly preferred embodiment the antimicrobial ion is silver.

The antimicrobial compound, particularly an antimicrobial metal ion exchange material, is preferably 0.1 to 5.0% by weight of the antimicrobial layer, and more preferably 0.5 to 3.0% by weight of the antimicrobial layer. It is preferred when the antimicrobial ion is silver, that the silver ion laydown is from 1 mg/m² to 1000 mg/m².

In a second embodiment the invention is a multilayer medium having antimicrobial properties comprising a support, an antimicrobial layer comprising a binder and an antimicrobial compound, or antimicrobial moiety thereof, which is released into the surrounding environment; and a diffusion layer; wherein the antimicrobial layer is between the support and the diffusion layer and wherein the diffusion layer changes the rate at which the antimicrobial compound is released from the antimicrobial layer into the surrounding environment. The antimicorbial layer, antimicorbial compound and diffusion layer are the same as described above.

To form the antimicrobial layer of the inventive article, or multilayer medium, the antimicrobial compound should be uniformally and homogeneously mixed within the binder. Mixing may be accomplished by a number of methods. For example, a copolymer or polymer and the AMC may be dispersed in a suitable solvent. The preferred solvent is water, although other solvents may be used. The process may include the addition of surfactants, peptizers, dispersion aids, etc. to facilitate the mixing. Alternatively the mixture may be formed by directly compounding the polymer and AMC at the melting temperature of the polymer as is done by screw compounding. Likewise, to form the diffusion layer, the polymer of the diffusion layer may be dissolved or dispersed in a vehicle.

When preparing the article of the invention, the antimicrobial layer and the diffusion layer may then be applied sequentially to the surface of an article via painting, brushing, spraying, blow-molding, blade coating, dip coating, etc. or the two layers may be applied simultaneously such as in multilayer curtain coating. The inventive article may also be formed by screw compounding the AMC in the binder and then co-extruding the antimicrobial layer and the diffusion layer together. Further, an adhesive may be applied to the surface of the antimicrobial layer and then fastened to an article via gluing, molding, lamination, etc.

The inventive article may comprise the surfaces of walls, counter tops, floors, furniture, textiles, consumer items, packaging, medical products such as bandages, prosthetics, etc. to prevent the growth of microbes such as bacteria, mold and yeast and to reduce the risk of the transmission of infectious disease. The inventive article may be prepared by many methods such as painting, spraying, casting, molding, blowing, coating, extruding, etc.

As noted above, the antimicrobial medium, preferably a film, comprises a support, an antimicrobial layer and a diffusion layer. Examples of supports useful for practice of the invention are resin-coated paper, paper, polyesters, or micro porous materials such as polyethylene polymer-containing material sold by PPG Industries, Inc., Pittsburgh, Pa. under the trade name of Teslin®, Tyvek® synthetic paper (DuPont Corp.), and OPPalyte® films (Mobil Chemical Co.) and other composite films listed in U.S. Pat. No. 5,244,861. Opaque supports include plain paper, coated paper, synthetic paper, photographic paper support, melt-extrusion-coated paper, and laminated paper, such as biaxially oriented support laminates. Biaxially oriented support laminates are described in U.S. Pat. Nos. 5,853,965; 5,866,282; 5,874,205; 5,888,643; 5,888,681; 5,888,683; and 5,888,714, the disclosures of which are hereby incorporated by reference. These biaxially oriented supports include a paper base and a biaxially oriented polyolefin sheet, typically polypropylene, laminated to one or both sides of the paper base. Transparent supports include glass, cellulose derivatives, e.g., a cellulose ester, cellulose triacetate, cellulose diacetate, cellulose acetate propionate, cellulose acetate butyrate; polyesters, such as poly(ethylene terephthalate), poly(ethylene naphthalate), poly(1,4-cyclohexanedimethylene terephthalate), poly(butylene terephthalate), and copolymers thereof; polyimides; polyamides; polycarbonates; polystyrene; polyolefins, such as polyethylene or polypropylene; polysulfones; polyacrylates; polyether imides; and mixtures thereof. The papers listed above include a broad range of papers, from high end papers, such as photographic paper to low end papers, such as newsprint. Another example of supports useful for practice of the invention are fabrics such as wools, cotton, polyesters, etc. Preferably the medium is flexible.

In a suitable embodiment the antimicrobial layer has a thickness in the range of 0.1 μm to 100 μm, and more preferably the thickness of said antimicrobial layer is about 1 μm to 10 μm. Generally the support has a thickness in the range of 0.025 mm to 5 mm. In a preferred embodiment utilizing an antimicrobial ion exchange material, wherein silver is the antimicrobial ion, the silver laydown is preferably from 1 mg/m² to 1000 mg/m². The multilayer medium may then be attached to the surface of an article to impart antimicrobial activity to that item. The diffusion layer should be placed such that it is the outermost surface of the article to maximize the control over the antimicrobial activity of that article. The medium may be attached by many means such as lamination, gluing, wrapping, etc. The medium may further comprise an adhesive layer on the opposite side of the support from the antimicrobial layer The following examples are intended to illustrate, but not to limit the invention.

### EXAMPLES

Preparation of Silver ion Sequester/Release Dispersion: Into a 1.0 L container was placed 100.00 g of amorphous Zr(HPO₄)₂.H₂O (from MEI corporation) in 200.0 g of distilled water. To this suspension was added slowly, (over 5′) 133 ml (146.3 g) of 2.5 M NaOH. The pH was 7.7 @ 34° C. Then, with stirring, were simultaneously added: 166 ml (208.3 g) of 1.5 M AgNO3 at 8.3 ml/min for 20 minutes and 330.0 ml (336.3 g) 0.25 M NaOH at 16.5 ml/min for 20 minutes. The pH was maintained at about 5.0 throughout the addition. The contents were then allowed to stir overnight @ 40° C. The final pH was 5.20. Silver analysis indicated the final dispersion to be 2.71 weight % Ag. The final silver ion sequester and release agent material composition was calculated to be Zr(H_{0.41}Ag_{0.37}Na_{0.22}PO₄)₂.H₂O.

### Example 1

Samples (E1-E7)

The experiments were performed by forming a coating solution of Zr(H_{0.41}Ag_{0.37}Na_{0.22}PO₄)₂.H₂O and the indicated polymer (Table 1) in an appropriate solvent. For PVA, water was used; for EVOH, a 50:50 mixture of water and isopropanol was used and for all others acetone was used as the solvent. The coating solution was then applied onto a clean plastic support using a doctor blade having a 125 micron gap, and dried to form a film. In each case, the thickness of the film was between 5 and 6 microns. A 5 cm×5 cm piece of this film was then immersed in 25.0 ml of aqueous 0.1 M NaNO₃, allowed to remain suspended there for the indicated time (Table 1), and the silver concentration in the aqueous medium was then determined by atomic emission spectroscopy.

TABLE 1
Percentage of antimicrobial silver ion released over time for
Samples (E1-E7)

| | | Permeability | Silver Lay | % Ag Release | | |
|---|---|---|---|---|---|---|
| | Polymer | coefficient, | down, | in time | | |
| Sample | or Resin | P × 10¹³ | (μg/cm²⁾ | 1 h | 1 d | 4 d |
| E1 | PVA | 42,000 | 1.4 | 90 | 100 | — |
| E2 | PVA | 42,000 | 6.9 | 32 | 36 | 70 |
| E3 | EVOHA | 10,000 | 1.2 | 40 | 65 | 100 |
| E4 | EVOH | 10,000 | 11.6 | 14 | 38 | 43 |
| E5 | CA | 5,500 | 12.0 | 2 | 15 | 21 |
| E6 | PMM | 480 | 28.7 | 2 | 7 | 7 |
| E7 | KYNAR | <5 | 27.4 | 0 | 1 | 6 |

The permeability coefficient is taken from S. Pauly in “Permeability and Diffusion Data”. PVA is polyvinyl alcohol, EVOH is polyethylene polyvinyl alcohol copolymer, CA is cellulose acetate, PMM is polymethylmethacrylate, KYNAR is poly(vinylidenefluoride-co-tetrafluoroethylene).

The results of Table 1 indicate that the exchange rate of antimicrobial silver to the surrounding medium is strongly dependent upon the water permeability of the polymer. The results show that coatings of antimicrobial materials in polymers having a high permeability of water may quickly reach the minimum inhibitory concentration of antimicrobial. However, the activity of such coatings will be short lived due to depletion of silver ion, and consumption of the silver ion by bacteria and other microbes. The results further show that coatings of antimicrobials in polymers having very low permeability to water have a much slower rate of exchange of the antimicrobial to the surrounding medium. For these coatings, a MIC may never be achieved, or may only be achieved very slowly, when the silver concentration (or laydown in Table 1) is very high. The data of Table 1 allow the careful design of antimicrobial articles, wherein the diffusion rate of the AMC may be suited to the requirements of the application, such as the desired laydown of the AMC and the longevity of the article. In this manner, the data of Table 1 may be used to accurately predict the binder and diffusion layer combination that is uniquely suited to the requirements of the application. For example, an antimicrobial article which requires antimicrobial activity over the period of seconds or minutes, would require both binder and diffusion layer having a high permeability to water, and a relatively low AMC concentration. Alternatively, an antimicrobial article which requires antimicrobial activity over the period of days or months, would require a diffusion layer with a low permeability to water, and a relatively high AMC concentration to slowly replenish the leached AMC. The utility of the invention becomes yet more apparent in the following examples.

### Example 2

Samples and Comparison Samples (C1, E8-E12)

The antimicrobial activity of the coatings, (C1, E8-E12), prepared as described above and as indicated in Table 2, were tested according to a method adapted from AATCC 100, 147; JIS Z 2801-2000; ASTM 2180-01. The principle of the test is to incubate a piece of coating that has a well-defined surface in a test-tube with a given volume of liquid growth medium, in which a well-defined amount of bacteria has been inoculated. The activity of the AMC will be measured by its effect on the number of viable bacteria after a given incubation time at a given temperature.

In this specific experiment the following operating conditions were applied. The surface of coating was 1×1 cm incubated in 1 ml of growth solution. The Trypcase Soy Broth growth medium was used diluted 1/10 in sterile water. This is a common growth medium used for the bacteria strain tested. Incubations were performed at 37° C. under aerobic conditions in the dark. Daily, over three days, aliquots of the solution were sampled and analyzed for bacteria number by the standard method of heterotrophic plate counts on Trypcase Soy Agar at 37° C. over 24 hours. Results are reported in Colony Forming Units/ml (CFU/ml).

The bacteria strain tested was Pseudomonas aeruginosa (ATCC 27853), which is commonly used as a representative of gram-negative bacteria in this kind of antimicrobial activity testing. Enough bacteria were inoculated in the test tube defined above in order to get an initial concentration of 100,000 bacteria/ml. After 1 day of incubation in the absence of antimicrobial compounds, the bacteria concentration in the solution was typically in the range of 10,000,000 to 100,000,000 CFU/ml. Given the operating conditions of the method, only concentrations of bacteria below 500,000,000 CFU/ml can be measured. When above this limit, results are expressed as >500,000,000 CFU/ml.

TABLE 2
Number of CFU/ml in the solution incubated with coatings for various
times at 37° C.

| Sample or | polymer and | | | | |
|---|---|---|---|---|---|
| Comparison | silver laydown | | CFU after 1 | CFU after 2 | CFU after 3 |
| Sample | (μg/cm²) | | days | days | days |
| E8 | EVOH | 1.2 | 584,000 | 696,000 | 93,000 |
| E9 | EVOH | 11.6 | 28,000 | 94,000 | 9,000 |
| C1 | KYNAR | 1.4 | >500,000,000 | 144,000,000 | >500,000,000 |
| E10 | KYNAR | 16.1 | 11,000 | 4,000 | 650 |
| E12 | PMM | 11.8 | 255,000 | 109,000 | 4,000 |
| E12 | CA | 19.4 | 34,000 | 13,000 | 490 |

The permeability coefficient is taken from S. Pauly in “Permeability and Diffusion Data”. PVA is polyvinyl alcohol, EVOH is polyethylene polyvinyl alcohol copolymer, CA is cellulose acetate, PMM is polymethylmethacrylate, KYNAR is poly(vinylidenefluoride-co-tetrafluoroethylene).

The data of Table 2 indicate that antimicrobial polymeric layers having high permeability to water achieve antimicrobial levels more quickly and require less AMC laydown. However, these materials begin to lose their effectiveness over time due to the rapid depletion of the AMC from the antimicrobial layer. Alternatively, antimicrobial polymeric layers having low permeability to water may form antimicrobial surfaces if the silver concentration is significantly higher, and further if the longevity of antimicrobial action is improved.

The invention has been described in detail with particular reference to the preferred embodiments thereof, but it will be understood that variations and modifications can be effected within the scope of the invention.

1. An article comprising on the surface thereof an antimicrobial layer comprising a binder and an antimicrobial compound, wherein said antimicorbial compound or an antimicrobial moiety thereof, is released into the surrounding environment; and a diffusion layer; wherein the antimicrobial layer is between the surface of the article and the diffusion layer and wherein the diffusion layer changes the rate at which the antimicrobial compound is released from the antimicrobial layer into the surrounding environment.

2. The article of claim 1 wherein said antimicrobial compound is a benzoic acid, sorbic acid, nisin, thymol, allicin, peroxide, imazalil, triclosan, benomyl, antimicrobial metal-ion exchange material, metal colloid, anhydride, or organic quaternary ammonium salt.

3. The article of claim 2 wherein said antimicrobial compound is an antimicrobial metal ion exchange material comprising a metal ion exchange material which has been exchanged or loaded with antimicrobial ions.

4. The article of claim 3 wherein said metal ion exchange material is zirconium phosphate, metal hydrogen phosphate, sodium zirconium hydrogen phosphate, zeolite, clay, an ion-exchange resin, an ion exchange polymer, porous alumino-silicate, a layered ion-exchange material or magnesium silicate.

5. The article of claim 3 wherein the antimicrobial ions are metal ions selected from silver, copper, nickel, zinc, gold and tin.

6. The article of claim 5 wherein said metal ion is silver.

7. The article of claim 1 wherein the diffusion layer has a water permeability that is greater than the water permeability of the antimicrobial layer.

8. The article of claim 1 wherein the diffusion layer has a water permeability that is less than the water permeability of the antimicrobial layer.

9. The article of claim 1 wherein the diffusion layer has a water permeability greater than 500 [(cm³ cm)/(cm²sec/Pa)]×10¹³.

10. The article of claim 1 wherein the diffusion layer comprises polyurethane, polyester, polyamide, polymethacrylate, polyethylene terephthalate, polyethylene-polyvinyl alcohol copolymer, polystyrene, ethyl cellulose, cellulose acetate, cellulose nitrate, polyethylene and polypropylene, nylon or polyacrylonitrile.

11. The article of claim 1 wherein the diffusion layer comprises polyurethane, polyester, polyamide, cellulose acetate, polymethacrylate, polystyrene, polypropylene, polyethylene-polyvinyl alcohol copolymer or polyethylene.

12. The article of claim 1 wherein the diffusion layer has a thickness in the range of 0.1 microns to 10.0 microns.

13. The article of claim 1 where the thickness of said diffusion layer is about 1.0 micron to 5.0 microns.

14. The article of claim 1 where the antimicrobial layer has a water permeability of greater than 5000 [(cm 3 cm)/(cm 2sec/Pa)]×10¹³.

15. The article of claim 1 wherein the binder of the antimicrobial layer is polyvinyl alcohol, cellophane, water-based polyurethanes, nylon, high nitrile resins, polyethylene-polyvinyl alcohol copolymer, polystyrene, ethyl cellulose cellulose acetate and cellulose nitrate, aqueous latexes, polyacrylic acid, and polystyrene sulfonate.

16. The article of claim 1 wherein the antimicrobial compound is 0.1 to 5.0% by weight of the antimicrobial layer.

17. The article of claim 3 wherein the antimicrobial metal ion exchange material is 0.5 to 3.0% by weight of the antimicrobial layer.

18. The article of claim 6 wherein the silver laydown is from 1 mg/m² to 1000 mg/m².

19. A multilayer medium having antimicrobial properties comprising a support, an antimicrobial layer comprising a binder and an antimicrobial compound, wherein said antimicrobial compound or an antimicrobial moiety thereof, is released into the surrounding environment; and a diffusion layer; wherein the antimicrobial layer is between the support and the diffusion layer and wherein the diffusion layer changes the rate at which the antimicrobial compound is released from the antimicrobial layer into the surrounding environment.

20. The medium of claim 19 wherein the support layer is made from one or more of the following:
resin-coated paper,
paper, polyesters,
micro porous materials
polyethylene
plain paper,
coated paper,
synthetic paper,
photographic paper support,
melt-extrusion-coated paper,
laminated paper,
biaxially oriented polyolefin
polypropylene
glass,
cellulose derivatives, or
polyesters.

21. The medium of claim 19 wherein the medium is flexible.

22. The medium of claim 19 wherein the support layer has a thickness in the range of 0.025 mm to 5 mm.

23. The medium of claim 19 further comprising an adhesive layer on the opposite side of the support from the antimicrobial layer.

24. The medium of claim 19 wherein said antimicrobial compound is a benzoic acid, sorbic acid, nisin, thymol, allicin, peroxide, imazalil, triclosan, benomyl, antimicrobial metal-ion exchange material, metal colloid, anhydride, or organic quaternary ammonium salt.

25. The medium of claim 24 wherein said antimicrobial compound is an antimicrobial metal ion exchange material comprising a metal ion exchange material which has been exchanged or loaded with antimicrobial ions.

26. The medium of claim 25 wherein said metal ion exchange material is zirconium phosphate, metal hydrogen phosphate, sodium zirconium hydrogen phosphate, zeolite, clay, an ion-exchange resin, an ion exchange polymer, porous alumino-silicate, a layered ion-exchange material or magnesium silicate.

27. The medium of claim 25 wherein the antimicrobial ions are metal ions selected from silver, tin, copper, nickel, zinc and gold.

28. The medium of claim 27 wherein said metal ion is silver.

29. The medium of claim 19 wherein the diffusion layer has a water permeability that is greater than the water permeability of the antimicrobial layer.

30. The medium of claim 19 wherein the diffusion layer has a water permeability that is less than the water permeability of the antimicrobial layer.

31. The medium of claim 19 wherein the diffusion layer has a water permeability greater than 500 [(cm³ cm)/(cm²sec/Pa)]×10¹³.

32. The medium of claim 19 wherein the diffusion layer comprises a polyurethane, polyester, polyamide, polymethacrylate, polyethylene terephthalate, polyethylene-polyvinyl alcohol copolymer, polystyrene, ethyl cellulose, cellulose acetate, cellulose nitrate, polyethylene and polypropylene, nylon or polyacrylonitrile.

33. The medium of claim 19 wherein the diffusion layer comprises polyurethane, polyester, polyamide, cellulose acetate, polymethacrylate, polystyrene, polypropylene, polyethylene-polyvinyl alcohol copolymer or polyethylene.

34. The medium of claim 19 wherein the diffusion layer has a thickness in the range of 0.1 microns to 10.0 microns.

35. The medium of claim 19 where the thickness of said diffusion layer is about 1.0 micron to 5.0 microns.

36. The medium of claim 19 where the antimicrobial layer has a water permeability of greater than 5000 [(cm³ cm)/(cm²sec/Pa)]×10¹³.

37. The medium of claim 19 wherein the binder of the antimicrobial layer is polyvinyl alcohol, cellophane, water-based polyurethanes, nylon, high nitrile resins, polyethylene-polyvinyl alcohol copolymer, polystyrene, ethyl cellulose cellulose acetate and cellulose nitrate, aqueous latexes, polyacrylic acid, or polystyrene sulfonate.

38. The medium of claim 19 wherein the antimicrobial compound is 0.1 to 5.0% by weight of the antimicrobial layer.

39. The medium of claim 38 wherein the antimicrobial metal ion exchange material is 0.5 to 3.0% by weight of the antimicrobial layer.

40. The medium of claim 28 wherein the silver laydown is from 1 mg/m² to 1000 mg/m².

41. The article of claim 1 further comprising a support between the article and the antimicrobial layer.
